(19) United States
(12) Patent Application Publication
(10) Pub. No.: US 2015/0104845 A1
(43) Pub. Date: Apr. 16, 2015

(54) ACOUSTIC MANIPULATION PROCESS AND ACOUSTIC MANIPULATION DEVICE

(71) Applicant: THE PENN STATE RESEARCH FOUNDATION, University Park, PA (US)

(72) Inventors: Tony Jun HUANG, State College, PA (US); Yuchao CHEN, State College, PA (US)

(21) Appl. No.: 14/514,108

(22) Filed: Oct. 14, 2014

Related U.S. Application Data
(60) Provisional application No. 61/891,010, filed on Oct. 15, 2013.

(51) Int. Cl. G01N 15/14; C12N 13/00

(52) U.S. Cl. CPC G01N 15/1404; C12N 13/00; G01N 15/1484; G01N 2015/142
USPC 435/173.9; 435/283.1; 210/748.05

(57) ABSTRACT

An acoustic manipulation process and acoustic manipulation device are disclosed. The acoustic manipulation process includes providing a device having a pathway positioned to receive a flow of a particle-containing fluid, transporting the particle-containing fluid into the pathway, and applying standing surface acoustic waves to the particle-containing fluid while the particle-containing fluid is flowing through the pathway. The applying of the standing surface acoustic waves to the particle-containing fluid includes nodes and anti-nodes of the standing surface acoustic waves extending through the particle-containing fluid. The acoustic manipulation device includes a pathway positioned to receive a flow of a particle-containing fluid, and a mechanism for generating and applying standing surface acoustic waves to the particle-containing fluid while the particle-containing fluid is flowing through the pathway.

### STATEMENT CONCERNING FEDERALLY-SPONSORED RESEARCH

The invention was developed under Grant No. ECCS0801922, awarded by the National Science Foundation and OD007209, awarded by the National Institute of Health. The U.S. Government has certain rights in this invention.

### FIELD OF THE INVENTION

The present invention is directed to processes and devices for the manipulation of particle-containing fluid. More particularly, the present invention is directed to acoustic manipulation.

### BACKGROUND OF THE INVENTION

The ability to enrich cells or other biological samples with high viability and recovery efficiency is important in many applications in bioanalysis and medical diagnostics. This ability is even more important when dealing with low-abundance cell types (for example, rare cells), such as circulating tumor cells, stem cells, and fetal cells.

Increasing the concentration of such low-abundance cells is important since higher sample concentration often leads to improved signal-to-noise ration analysis. Conventional techniques for increasing the concentration of such low-abundance cells, such as centrifugation, suffer from drawbacks of significant loss cell viability, limitations on quantities capable of being recovered, and/or low recovery efficiency.

Microfluidic techniques are known for transporting fluids. However, use of microfluidics in conjunction with acoustic fields for separation and/or concentration of particles in particle-containing fluids has not been previously practicable due to the high cost of piezoelectric materials.

An acoustic manipulation process and acoustic manipulation device that show one or more improvements in comparison to the prior art would be desirable in the art.

### BRIEF DESCRIPTION OF THE INVENTION

In an embodiment, an acoustic manipulation process includes providing a device having a pathway positioned to receive a flow of a particle-containing fluid, transporting the particle-containing fluid into the pathway, and applying standing surface acoustic waves to the particle-containing fluid while the particle-containing fluid is flowing through the pathway. The applying of the standing surface acoustic waves to the particle-containing fluid includes nodes and anti-nodes of the standing surface acoustic waves extending through the particle-containing fluid.

In another embodiment, an acoustic manipulation process includes providing a device having microtubes removably positioned in contact with a coupling gel on a substrate and configured to receive a flow of suspension containing cells, transporting the suspension through the microtubes, and applying standing surface acoustic waves from interdigital transducers on the substrate to the suspension while the suspension is flowing through the microtubes in a direction parallel with the applying of the standing surface acoustic waves. The applying of the standing surface acoustic waves to the suspension includes nodes and anti-nodes of the standing surface acoustic waves extending through the suspension. The applying of the standing surface acoustic waves manipulates the cells, thereby decreasing the concentration of the cells in the suspension while the suspension flows through the microtubes.

In another embodiment, an acoustic manipulation device includes a pathway positioned to receive a flow of a particle-containing fluid, and a mechanism for generating and applying standing surface acoustic waves to the particle-containing fluid while the particle-containing fluid is flowing through the pathway.

Other features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 is a perspective view of an embodiment of an acoustic manipulation device during an embodiment of an acoustic manipulation process, according to the disclosure.

FIG. 2 is a side view of an embodiment of an acoustic manipulation device having a coupling material, according to the disclosure.

FIG. 3 is a schematic top view of a pathway for an embodiment of an acoustic manipulation device showing enrichment regions of higher particle concentration within a particle-containing fluid, according to the disclosure.

FIG. 4 is a schematic top view of multiple pathways for an embodiment of an acoustic manipulation device, according to the disclosure.

FIG. 5 is a fluorescence image corresponding to a sectioned view of the multiple pathways in FIG. 4 illustrating enrichment of particles within an embodiment of the acoustic manipulation device, according of the disclosure.

FIG. 6 is a fluorescence image corresponding to a sectioned view of an interface along a pathway between an entrance region with no coupling gel and a region with a coupling gel in an embodiment of an acoustic manipulation device, according to the disclosure.

FIG. 7 is a fluorescence image corresponding to a sectioned view of a region with a coupling gel in an embodiment of an acoustic manipulation device, according to the disclosure.

FIG. 8 is a fluorescence image corresponding to a sectioned view of an interface along a pathway between a region with a coupling gel and an exit region with no coupling gel in an embodiment of an acoustic manipulation device, according to the disclosure.

FIG. 9 a graphical representation of enrichment, saturation, and release using an embodiment of an acoustic manipulation device according to an embodiment of an acoustic manipulation process, according to the disclosure.

FIG. 10 is a fluorescence image corresponding to a sectioned view of an interface along a pathway between an entrance region with no coupling gel and a region with a coupling gel in an embodiment of an acoustic manipulation device prior to a standing surface acoustic wave being applied, according to the disclosure.

FIG. 11 is a fluorescence image corresponding to a sectioned view of an interface along a pathway between an entrance region with no coupling gel and a region with a coupling gel in an embodiment of an acoustic manipulation device after a standing surface acoustic wave is applied and during enrichment, according to the disclosure.

FIG. 12 is a fluorescence image corresponding to a sectioned view of an interface along a pathway between an entrance region with no coupling gel and a region with a coupling gel in an embodiment of an acoustic manipulation device after a standing surface acoustic wave is applied and during enrichment, according to the disclosure.

FIG. 13 is a fluorescence image corresponding to a sectioned view of an interface along a pathway between an entrance region with no coupling gel and a region with a coupling gel in an embodiment of an acoustic manipulation device after a standing surface acoustic wave is applied, after enrichment, and during saturation, according to the disclosure.

FIG. 14 is a fluorescence image corresponding to a sectioned view of an interface along a pathway between an entrance region with no coupling gel and a region with a coupling gel in an embodiment of an acoustic manipulation device after a standing surface acoustic wave is applied, after enrichment, and during saturation, according to the disclosure.

FIG. 15 is a fluorescence image corresponding to a sectioned view of an interface along a pathway between an entrance region with no coupling gel and a region with a coupling gel in an embodiment of an acoustic manipulation device after a standing surface acoustic wave is applied, after enrichment, after saturation, and during release, according to the disclosure.

FIG. 16 shows a plot of fluorescence intensity relating to several input powers over a period of time corresponding to an embodiment of using an acoustic manipulation device, according to the disclosure.

FIG. 17 shows a plot of fluorescence intensity relating to several flow rates over a period of time corresponding to an embodiment of using an acoustic manipulation device, according to the disclosure.

FIG. 18 shows a plot of absorbance at 450 nm for several processes/treatments in comparison to transport through an embodiment of an acoustic manipulation device, according to the disclosure.

Wherever possible, the same reference numbers will be used throughout the drawings to represent the same parts.

### DETAILED DESCRIPTION OF THE INVENTION

Provided are an acoustic manipulation process and an acoustic manipulation device. Embodiments of the present disclosure, for example, in comparison to concepts failing to include one or more of the features disclosed herein, allow enrichment/concentration of particles in particle-containing fluids (for example, cells in blood), permit broader range of materials to be used for microchannels, permit simpler and less expensive manufacture/fabrication of devices for particle manipulation/concentration, permit greater recovery efficiency of particles within particle-containing fluids, permit study of low-abundance cells not previously able to be practically studied, permit enhanced travel of acoustic waves, permit concentration/separation without physical contact (for example, being non-invasive and/or label-free), are compatible with biomedical and/or bioanalytical applications, permit other suitable advantages and distinctions, and/or a combination thereof.

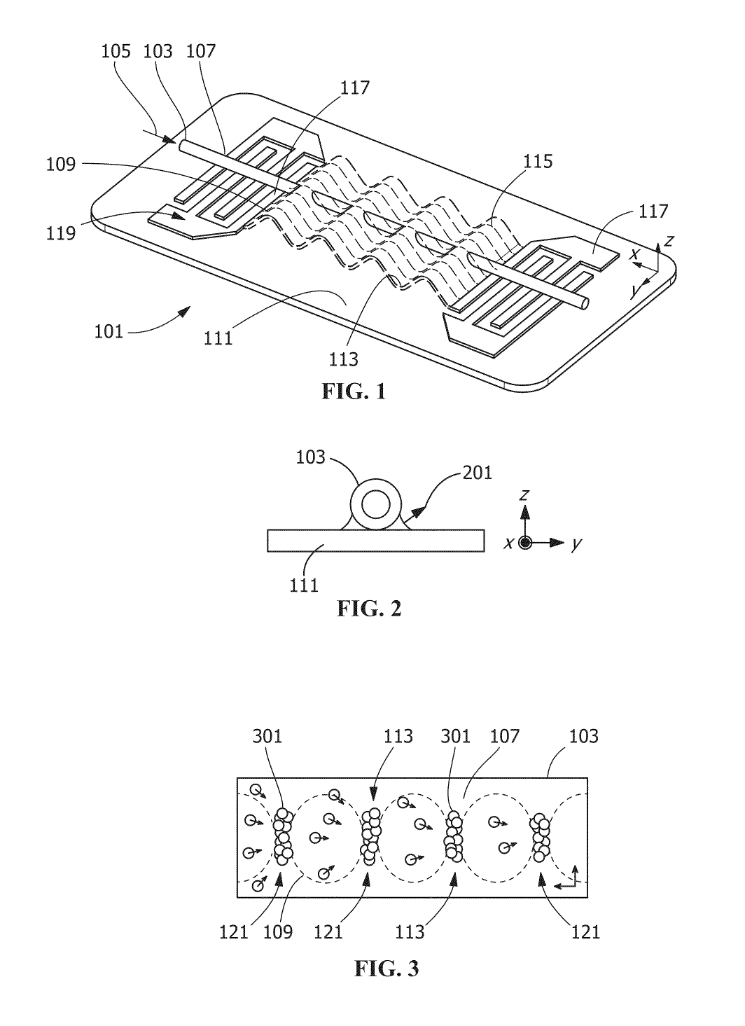

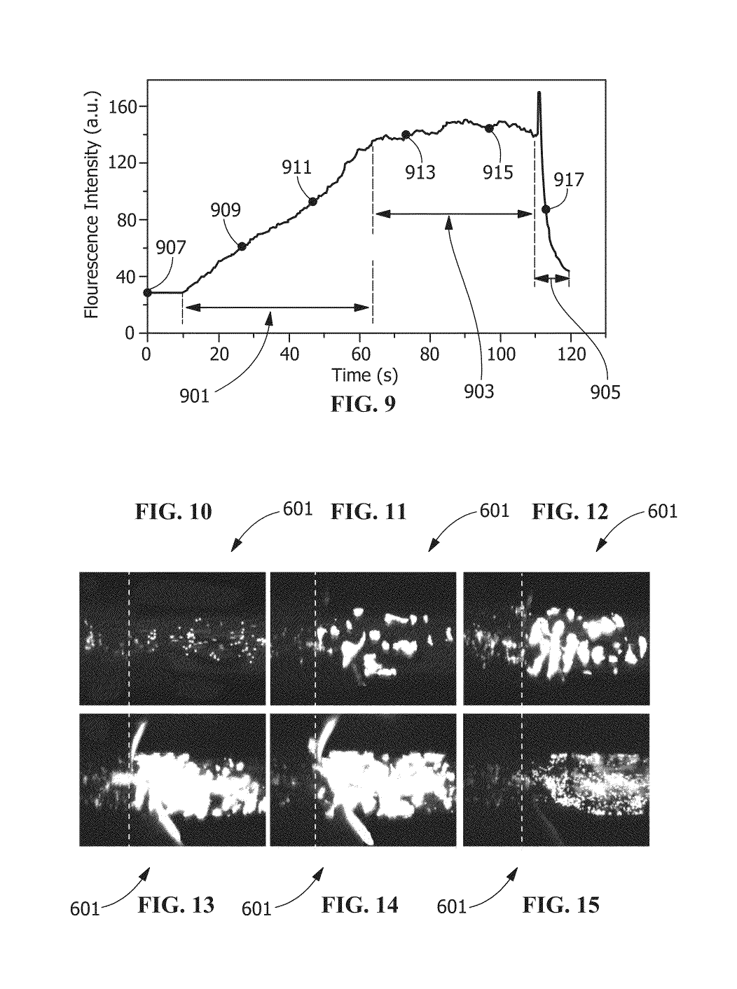

Referring to FIG. 1, in one embodiment, an acoustic manipulation process is performed using an acoustic manipulation device 101. As illustrated by FIG. 9, in one embodiment, the acoustic manipulation process includes enrichment 901, saturation 903, and release 905. The enrichment 901 includes the use of the acoustic manipulation device to aggregate particles 301 (see FIG. 3) within a pathway 103 (see FIG. 1) from flow of a particle-containing fluid 107 (see FIG. 1). The saturation 903 occurs after concentration reaches a saturation concentration. The release 905 includes deactivation of the acoustic manipulation device 101 and, thus, release of the particles 301. In a further embodiment, between the saturation 903 and the release 905, a purification using a washing buffer is applied, for example, to remove contaminated molecules and exchange mediums, such as in biochemical analysis. The purification occurs during a substantially stable portion of the saturation 903.

Referring again to FIG. 1, the acoustic manipulation device 101 includes the pathway 103 positioned to receive a flow 105 of the particle-containing fluid 107. In the acoustic manipulation process, the particle-containing fluid 107 is transported into the pathway 103 and standing surface acoustic waves 109 are applied to the particle-containing fluid 107, for example, with the pathway 103 being oriented in the direction of or substantially in the direction of the propagation direction and within an activation region for the standing surface acoustic waves 109. In a further embodiment, the standing surface acoustic waves 109 are applied while the particle-containing fluid 107 is flowing through the pathway 103. All or a portion of the particle-containing fluid 107 is trapped or flows from the pathway 103.

The pathway 103 is any suitable channel(s), trough(s), tube(s), microtube(s), pipe(s), micropipe(s), hose(s), or combination thereof, capable of permitting the flow of the particle-containing fluid 107. The pathway 103 is linear, substantially linear, or non-linear. In one embodiment, the pathway 103 includes a plurality of channels or other similar structures capable of permitting the flow of the particle-containing fluid 107. Such channels or other similar structures are parallel, substantially parallel, converging, diverging, or not-parallel.

Suitable materials for the pathway 103 include, but are not limited to, polymeric materials (for example, polyethylene, polydimethylsiloxane, or other suitable long-chain carbon materials), plastic materials, ceramic materials, silicon, glass, quartz, biocompatible materials, or a combination thereof.

Suitable dimensions for the pathway 103 include an inner diameter of between 20 micrometers and 10,000 micrometers, between 50 micrometers and 2,000 micrometers, between 100 micrometers and 400 micrometers, between 150 micrometers and 350 micrometers, between 200 micrometers and 300 micrometers, between 270 micrometers and 290 micrometers, between 275 micrometers and 285 micrometers, between 278 micrometers and 282 micrometers, between 279 micrometers and 281 micrometers, 280 micrometers, or any suitable combination, sub-combination, range, or sub-range therein.

The flow 105 of the particle-containing fluid 107 occurs for any suitable period capable of manipulating/separating particles 301 (see FIG. 3). Suitable periods include, but are not limited to, at least 1 second, at least 10 seconds, at least 1 minute, at least 3 minutes, at least 6 minutes, at least 9 minutes, between 5 minutes and 30 minutes, between 8 minutes and 15 minutes, for 10 minutes, or any suitable combination, sub-combination, range, or sub-range therein.

In one embodiment, the flow 105 of the particle-containing fluid 107 is achieved by applying a pressure differential and/or pump mechanism. For example, in one embodiment, a syringe pump controls the velocity and the acceleration of the flow 105. Higher flow rates correspond with larger viscous drag forces on the particles 301 within the particle-containing fluid 107 leading to a decrease in recovery efficiency. The velocity of the flow 105 corresponds with such relationships, for example, by operating based upon an input power and a flow rate.

Suitable input powers include, but are not limited to, between 16 dBm and 20 dBm, between 17 dBm and 19 dBm, between 17 dBm and 18 dBm, between 18 dBm and 19 dBm or any suitable combination, sub-combination, range, or sub-range therein. Suitable flow rates include, but are not limited to, between 1 microliter per minute and 20 microliters per minute, between 1 microliter per minute and 15 microliters per minute, between 5 microliters per minute and 20 microliters per minute, between 5 microliters per minute and 15 microliters per minute, at 5 microliters per minute, at 7 microliters per minute, at 10 microliters per minute, at less than 15 microliters per minute, at less than 20 microliters per minute, or any suitable combination, sub-combination, range, or sub-range therein.

In one embodiment, the flow 105 of the particle-containing fluid 107 within the pathway 103 is on a scale permitting more precise control, for example, by being at a cellular level, thereby increasing capture efficiency and isolation purity. Such increased capture efficiency and isolation purity permits subsequent next-stage analysis (for example, genomic analysis, proteomic analysis, pharmaceutical screening, large molecular or small molecular detection, or a combination thereof). In a further embodiment, one or more known intermediate procedures used for macro-scale systems are eliminated from such next-stage analysis.

The particle-containing fluid 107 is any suitable medium with particles 301 (see FIG. 3) capable of being separated by acoustic manipulation. The particles 301 are cells (for example, rare cells, such as circulating tumor cells, stem cells, and fetal cells), or any other solid, semi-solid, and/or insoluble material. The medium is a suspension or any other fluid capable of having entrained particles.

In one embodiment, the particle-containing fluid 107 is or includes serially diluted human whole blood. Blood cells within the blood are at concentrations, such as between 1 cell per microliters and 200 cells per microliters, between 50 cells per microliters and 120 cells per microliters, between 90 cells per microliters and 110 cells per microliters, between 100 cells per microliters and 110 cells per microliters, between 101 cells per microliters and 108 cells per microliters, between 102 cells per microliters and 106 cells per microliters, between 103 cells per microliters and 105 cells per microliters, 103 cells per microliters, 104 cells per microliters, 105 cells per microliters, or any suitable combination, sub-combination, range, or sub-range therein.

The applying of the standing surface acoustic waves 109 manipulates the particles 301 entrained within the particle-containing fluid 107. For example, in one embodiment, the particles 301 (such as, blood cells) gradually accumulate at pressure nodes within one or more of the enrichment regions 121 within the pathway 103, resulting in concentrated zones of the particles 301. With recovery efficiency being the percent of measured concentration of the particles 301 to a theoretical 100% recovered concentration of the particles 301, suitable recovery efficiencies include, but are not limited to, between 90.2% and 96%, between 92.1% and 100%, between 87.8% and 100%, or any suitable combination, sub-combination, range, or sub-range therein.

The volume of the pathway 103, the flow rate of the particle-containing fluid 107, and the recovery efficiency are all related. In one embodiment, the volume of the pathway 103 is between 0.2 microliters and 0.4 microliters (for example, 0.3 microliters, based upon a length of 5 millimeters and a diameter of 0.28 millimeters), the volume of the particle-containing fluid 107 enriched is 70 microliters (based upon 10 minutes of the flow at a rate of 7 microliters per minute), and the recovery efficiency is above 90%. In further embodiments, the enrichment enhances the concentration of the particles 301 by a concentration factor of greater than 10, greater than 100, greater than 200, greater 500, or greater than 1,000. In some embodiments, the enrichment effectively traps and/or saturates the particles 301 of the particle-containing fluid 107 within the pathway 103 until the standing surface acoustic waves 109 are no longer provided.

The enrichment region(s) 121 is/are a distance of less than one wavelength (for example, the distance between two of the nodes 113 that are adjacent or a quantified distance, such as between 10 and 300 micrometers or about 100 micrometers). In one embodiment, the enrichment regions 121 include a distance of about one-half of the wavelength, between one-quarter and one-half of the wavelength, or less than one-quarter of the wavelength. Additionally or alternatively, in some embodiments, the pathway 103 includes a number of the enrichment regions 121, for example, more than 3, more than 10, more than 50, more than 100, more than 300, more than 500, between 10 and 500, between 30 and 300, between 40 and 100, between 40 and 80, between 40 and 60, or any suitable combination, sub-combination, range, or sub-range therein. In a further embodiment, regions outside of the enrichment regions 121 but within the pathway 103 are substantially devoid or devoid of the particles 301.

Although not intending to be bound by theory, it is believed that the standing surface acoustic waves 109 enrich particle concentration of the particle-containing fluid 107 by applying a pressure field to produce forces in an x-y plane that is parallel to a substrate 111. The forces are primary acoustic radiation force and viscous drag force and can be represented as follows:

$$\begin{matrix}{F}_{r} = - ( \frac{π {p}_{0}^{2} {V}_{p} {β}_{m}}{2 λ} ) φ ( β , ρ ) sin ( \frac{4 π x}{λ} ) & ( Equation 1 ) \\ φ = \frac{5 {ρ}_{p} - 2 {ρ}_{m}}{2 {ρ}_{p} + {ρ}_{m}} - \frac{{β}_{p}}{{β}_{m}} & ( Equation 2 ) \\ {F}_{v} = - 6 πη rv & ( Equation 3 )\end{matrix}$$

where p₀, Vₚ, λ, k, x, ρₘ, ρₚ, βₘ, βₚ, η, r and ν are pressure amplitude, particle volume, the standing surface acoustic wavelength, wave vector, distance from the pressure node, density of medium, density of cells, compressibility of medium, compressibility of cells, medium viscosity, cell radius, and relative velocity, respectively.

As shown in FIG. 3, a primary radiation force moves the particles 301 to the pressure nodes, for example, nodes 113 and/or anti-nodes 115. As the distance between the particles 301 decreases, a secondary radiation force plays a dominant role in aggregating the particles 301 together and forming an array of clusters within the enrichment regions 121. For example, in one embodiment, a component of the primary force along the x axis immobilizes the clusters in the pressure nodes by competing with the viscous drag force in the opposite direction. The clusters continue to attract nearby particles, growing in size and resulting in an increase in radiation forces on the clusters. Because each pressure node has a maximum trapping capacity, saturation occurs gradually from the upstream end to the downstream end of the pathway 103. When the volume of a trapped cluster is saturated, some of the particles 301 are flushed off and trapped again at the downstream pressure nodes and/or enrichment regions 121.

Use of the standing surface acoustic waves 109 for enrichment of cells as the particles 301 in the particle-containing fluid 107 permits viable cells to be concentrated at greater rates than previous techniques, such as, centrifugation. For example, in one embodiment, the standing surface acoustic waves 109 have little or no effect on the viability of the cells within the particle-containing fluid.

The standing surface acoustic waves 109 include the nodes 113 and the anti-nodes 115 that extend through the particle-containing fluid 107 and are generated by any suitable piezoelectric elements 117. The piezoelectric elements 117 are positioned directly or indirectly in contact with the substrate 111 that is directly or indirectly in contact with or defines the pathway 103, the substrate 111 being any suitable rigid, planar or substantially planar member, such as a LiNbO₃ substrate.

In one embodiment, the piezoelectric elements 117 are positioned perpendicular in one plane (for example, the y-z plane) and parallel in another plane (for example, the x-z plane) to the pathway 103. The piezoelectric elements 117 vibrate the substrate 111, the pathway 103, and the particle-containing fluid 107. Although not intending to be bound by theory, it is believed that the vibration along with the nodes 113 and the anti-nodes 115 decrease the concentration of the particles 301 in the particle-containing fluid 107 while the particle-containing fluid 107 flows through the pathway 103, for example, by trapping at least a portion of the particles 301 in the pathway 103. Upon being trapped, the particles 301 are capable of subsequently being recovered.

In one embodiment, the standing surface acoustic waves 109 are generated by alternating current (AC) signals being applied to the piezoelectric elements 117, for example, interdigital transducers and, thus, a non-uniform pressure field having a periodic distribution of the nodes 113 and anti-nodes 115 within the pathway 103. In a further embodiment, the AC signals are generated by a radiofrequency generator (such as, E4422B from Agilent Technologies, Santa Clara, Calif.) and amplified with a power amplifier (such as, 100A250A, from Amplifier Research, Souderton, Pa.) to form a one-dimensional standing surface acoustic wave field (not shown).

The piezoelectric elements 117 are formed or attached to the substrate 111 by any suitable process. In one embodiment, a pattern 119 of one or more of the piezoelectric elements 117 is developed through standard photolithography processes. For example, after depositing a metal double-layer (for example, Cr/Au, 50 Å/500 Å) with an electron-beam evaporator, the pattern 119 is formed on the substrate 111 by a lift-off process. The pattern 119 includes a number of electrodes, defined widths, and defined gaps.

In one embodiment, the number of electrodes in the pattern 119 is between 10 and 60, between 20 and 100, at least 10, at least 20, 20, or any suitable combination, sub-combination, range, or sub-range therein.

In one embodiment, defined widths in the pattern 119 are within a tolerance of 5 micrometers, 1 micrometer, 0.5 micrometers, 0.2 micrometers, or 0.1 micrometers.

In one embodiment, the defined gaps in the pattern 119 are between 1 and 100 micrometers, between 30 and 80 micrometers, between 40 and 60 micrometers, between 55 and 65 micrometers, at least 3 micrometers, at least 10 micrometers, at least 20 micrometers, at least 30 micrometers, at least 40 micrometers, at least 50 micrometers, 50 micrometers, or any suitable combination, sub-combination, range, or sub-range therein.

The piezoelectric elements 117 generate the standing surface acoustic waves 109 within a wavelength range and a resonance frequency range. In one embodiment, the wavelength range is between 50 micrometers and 1,000 micrometers, between 100 micrometers and 400 micrometers, between 150 micrometers and 250 micrometers, between 180 micrometers and 220 micrometers, between 190 micrometers and 210 micrometers, between 195 micrometers and 205 micrometers, between 199 micrometers and 201 micrometers, 200 micrometers, or any suitable combination, sub-combination, range, or sub-range therein.

In one embodiment, the resonance frequency range is between 5 kHz and 100 MHz, between 1 MHz and 700 MHz, between 5 MHz and 40 MHz, between 15 MHz and 25 MHz, between 18 MHz and 22 MHz, between 19 MHZ and 21 MHz, between 19 MHz and 20 MHz, between 19.4 MHz and 19.8 MHz, between 19.5 MHz and 19.7 MHz, 19.6 MHz, or any suitable combination, sub-combination, range, or sub-range therein.

Referring to FIG. 2, in one embodiment, the pathway 103 is removably secured to the substrate 111. The removable securing is by a coupling material 201 (for example, a coupling gel, such as, a glyercin-hydroxyethyl-cellulose material), by a releasable and mechanical securing mechanism (not shown), or by a combination thereof. In an embodiment with the coupling material, the pathway 103 and the substrate 111 are both in direct contact with the coupling material 201. In one embodiment, the coupling material 201 extends along the pathway 103 on the substrate 111 throughout or at least in a portion of an enrichment region 121 of the pathway 103, where manipulation and/or concentration of the particles 301 in the particle-containing fluid 107 occurs.

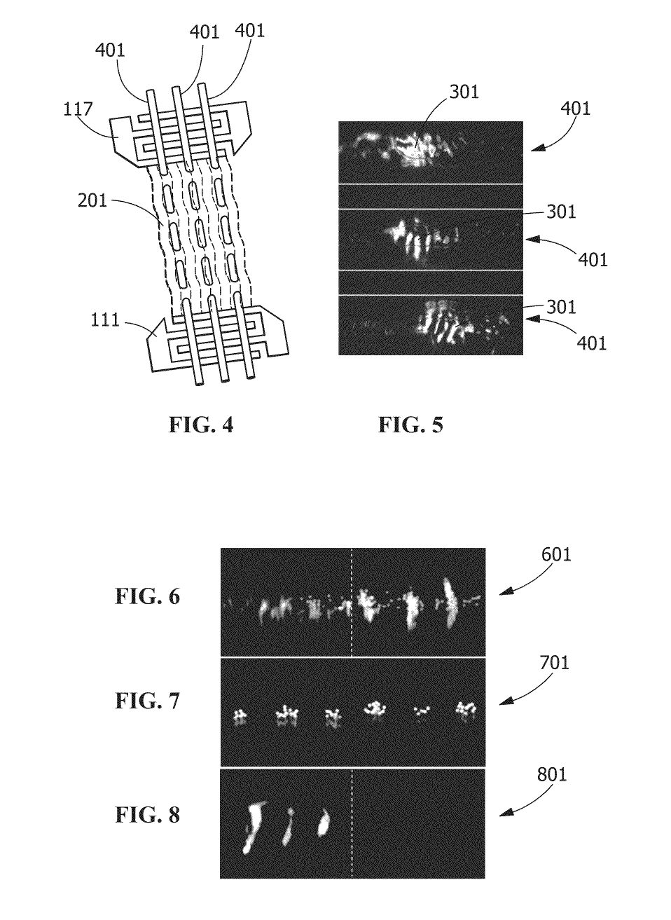

The removable securing of the pathway 103 to the substrate 111 permits the acoustic manipulation process to include removing of at least a portion of the pathway 103 from the acoustic manipulation device 101 and/or replacing the portion with at least a portion of a replacement pathway (not shown) directly or indirectly in contact with the substrate 111. Removal of the pathway 103 permits reduction or elimination of cross-contamination (for example, by being single-use) and/or permits additional configurations (for example, multiple parallel channels as shown in FIG. 4 capable of concurrently separating incompatible fluids as illustrated by the fluorescent image in FIG. 5). Removal of the pathway 103 permits the pathway 103 to be used as a container for the particles 301 trapped by the acoustic manipulation process, thereby allowing the particles 301 (for example, viable cells) to be analyzed at concentrations not previously attainable without use of a centrifuge. In one embodiment, the pathway 103 is sealed onsite, for example, by heat-sealing, and sent to an offsite location for analysis.

### EXAMPLES

In a first example, acoustic manipulation with a coupling gel positioned between the substrate 111 and the pathway 103 is compared to no coupling gel being positioned between the substrate 111 and the pathway 103. The standing surface acoustic waves 109 are applied along the substrate 111 while a fluid containing polystyrene beads flows through the pathways.

FIG. 6 shows a first section 601 that is at an interface along the pathway 103 between an entrance region with no coupling gel and the region with the coupling gel. As shown, the beads aggregate along the pressure nodes. FIG. 7 shows a second section 701 along the pathway 103 that is entirely within the region with the coupling gel. As shown, the beads are even more closely aggregated along the pressure nodes. FIG. 8 shows a third section 801 along the pathway 103 at the interface between the region with the coupling gel and an exit region with no coupling gel. As shown, the aggregation of the beads retains the beads within the pathway 103 and the exit region includes few or none of the beads.

In a second example, intensity of fluorescence of the polystyrene beads in the first example illustrates stages of an acoustic manipulation process. Data representative of enrichment, saturation, and release of the polystyrene beads are shown in FIG. 9.

FIG. 10 shows the first section 601 with the beads passing through at a consistent velocity prior to power being applied to the device 101, resulting in little or no change of the fluorescence intensity, corresponding with a first data point 907 on FIG. 9. FIG. 11 shows the first section 601 after the acoustic manipulation device 101 is used to apply the standing surface acoustic wave 109, resulting in an increase in fluorescence intensity, corresponding with a second data point 909 on FIG. 9. FIG. 12 shows the first section 601 after continuing to apply the standing surface acoustic wave 109, resulting in a substantially linear increase in fluorescence intensity, corresponding with a third data point 911 on FIG. 9. FIG. 13 shows the first section 601 after a saturation point is reached, resulting in a constant amount of the beads being retained but additional beads flowing through the pathway 103, corresponding with a fourth data point 913 on FIG. 9. FIG. 14 shows the first section 601 after continuing to apply the standing surface acoustic wave 109 beyond the saturation point, resulting in even more beads flowing through the pathway 103, corresponding with a fifth data point 915 on FIG. 9. FIG. 15 shows the release of the beads in the first section 601 corresponding to a spike in fluorescence intensity resulting from the beads flowing through a detection region to a substantial decay of the intensity indicating the release of the beads corresponding with a sixth data point 917 shown in FIG. 9.

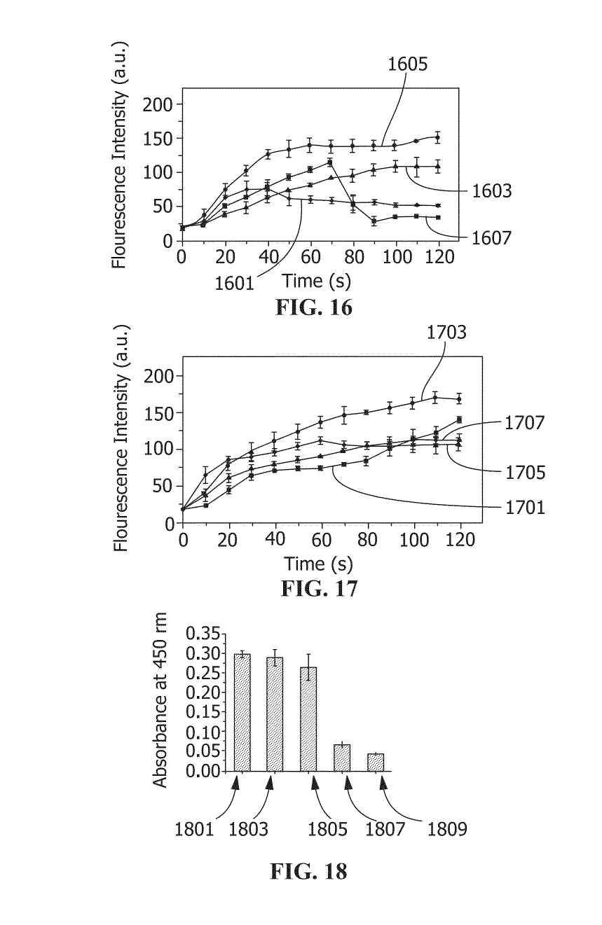

In a third example, intensity of fluorescence of the polystyrene beads in the first example illustrates the effect of various power levels for an acoustic manipulation process. As shown in FIG. 16, data representative of a first input power 1601 (specifically, 16 dBm), a second input power 1603 (specifically, 17 dBm), a third input power 1605 (specifically, 18 dBm), and a fourth input power 1607 (specifically, 19 dBm) is collected over a period of 120 seconds. The second input power 1603 and the third input power 1605 maintain the fluorescence intensity throughout the period, while the first input power 1601 and the fourth input power 1607 significantly decrease in fluorescence intensity over the same period, suggesting acoustic streaming becoming dominant and a failure of particle trapping.

In a fourth example, intensity of fluorescence of the polystyrene beads in the first example illustrates the effect of various power levels for an acoustic manipulation process. As shown in FIG. 17, data representative of a first flow rate 1701 (specifically, 5 microliters per minute), a second flow rate 1703 (specifically, 10 microliters per minute), a third flow rate 1705 (specifically, 15 microliters per minute), and a fourth flow rate 1707 (specifically, 20 microliters per minute) is collected over a period of 120 seconds. The first flow rate 1701 and the second flow rate 1703 show continued growth in fluorescence intensity. The third flow rate 1705 and the fourth flow rate 1707 show saturation based upon the fluorescence intensity decreasing by approximately 42 percent in comparison to the second flow rate 1703, presumably due to stronger viscous drag forces and increased momentum enabling the beads to escape the force from the standing surface acoustic wave 109.

In a fifth example, viability of cells travelling through the pathway 103 with the standing surface acoustic wave 109 being applied is collected. Specifically, microspheres with a diameter of 7 micrometers are suspended in a 1% sodium dodecyl sulfate solution at a concentration of 106 particles per microliter. Human whole blood is diluted with phosphate buffered saline solution at a concentration between 103 cells per microliter and 105 cells per microliter. Cell viability is compared with a first trial 1801 having no processing/treatment, a second trial 1803 having the standing surface acoustic wave 109 applied during travel through the pathway 103, a third trial 1805 without the standing surface acoustic wave 109 applied during travel through the pathway 103, a fourth trial 1807 with incubating at 65° C. for 15 minutes, and a fifth trial 1809 of a culture medium with no cells. As shown in FIG. 18, the first trial 1801, the second trial 1803, and the third trial 1805 show significantly higher absorbance than the fourth trial 1807 and the fifth trial 1809.

While the invention has been described with reference to one or more embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In addition, all numerical values identified in the detailed description shall be interpreted as though the precise and approximate values are both expressly identified.

What is claimed is:

1. An acoustic manipulation process, comprising:
providing an acoustic manipulation device having a pathway positioned to receive a flow of a particle-containing fluid;
transporting the particle-containing fluid into the pathway; and
applying standing surface acoustic waves to the particle-containing fluid while the particle-containing fluid is flowing through the pathway;
wherein the applying of the standing surface acoustic waves to the particle-containing fluid includes nodes and anti-nodes of the standing surface acoustic waves extending through the particle-containing fluid.

2. The process of claim 1, wherein the flowing of the particle-containing fluid occurs for at least 5 seconds.

3. The process of claim 1, wherein the flowing of the particle-containing fluid occurs in a direction parallel with the applying of the standing surface acoustic waves.

4. The process of claim 1, wherein the pathway for the transporting of the particle-containing fluid into the device includes a plurality of channels.

5. The process of claim 4, wherein the plurality of channels includes one or more tubes.

6. The process of claim 5, wherein the one or more tubes includes at least one microtube having a diameter of between 20 micrometers and 2,000 micrometers.

7. The process of claim 1, wherein the pathway is positioned directly or indirectly on a substrate.

8. The process of claim 7, wherein the substrate is a piezoelectric substrate.

9. The process of claim 7, wherein the pathway is removably secured to the substrate.

10. The process of claim 9, wherein a coupling material is in contact with the substrate and the pathway.

11. The process of claim 10, wherein the coupling material includes a coupling gel.

12. The process of claim 7, further comprising removing at least a portion of the pathway from the device and positioning the portion with at least a portion of a replacement pathway directly or indirectly in contact with the substrate.

13. The process of claim 1, wherein the particle-containing fluid is a fluid suspension.

14. The process of claim 1, wherein the applying of the standing surface acoustic waves is by interdigital transducers.

15. The process of claim 14, wherein the interdigital transducers are positioned on a substrate, the substrate directly or indirectly contacting the pathway, and the interdigital transducers vibrate the substrate.

16. The process of claim 1, wherein the applying of the standing surface acoustic waves manipulates particles within the particle-containing fluid, thereby decreasing the concentration of the particles in the particle-containing fluid while the particle-containing fluid flows through the pathway.

17. The process of claim 16, wherein the decreasing of the concentration of the particles in the particle-containing fluid while the particle-containing fluid flows through the pathway traps at least a portion of the particles in the pathway.

18. The process of claim 1, wherein particles in the particle-containing fluid are cells.

19. An acoustic manipulation process, comprising:
providing a device having microtubes removably positioned in contact with a coupling gel on a substrate and configured to receive a flow of a suspension containing cells;
transporting the suspension through the microtubes; and
applying standing surface acoustic waves from acoustic transducers on the substrate to the suspension while the suspension is flowing through the microtubes in a direction parallel with the applying of the standing surface acoustic waves;
wherein the applying of the standing surface acoustic waves to the suspension includes nodes and anti-nodes of the standing surface acoustic waves extending through the suspension;
wherein the applying of the standing surface acoustic waves manipulates the cells, thereby decreasing the concentration of the cells in the suspension while the suspension flows through the microtubes.

20. An acoustic manipulation device, comprising:
a pathway positioned to receive a flow of a particle-containing fluid; and
a mechanism for generating and applying standing surface acoustic waves to the particle-containing fluid while the particle-containing fluid is flowing through the pathway.
